(19) United States
(12) Patent Application Publication
(10) Pub. No.: US 2007/0276145 A1
(43) Pub. Date: Nov. 29, 2007

(54) Method of Preparing a Ring Compound Having Two Adjacent Chiral Centers

(75) Inventors: Lisa M. Schultze, Woodinville, WA (US); John Demattei, Braintree, MA (US); Brad Barnett, Northglenn, CO (US); Anthony Piscopio, Longmont, CO (US); Paul Nichols, Longmont, CO (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: ICOS CORPORATION, Bothell, WA (US)

(21) Appl. No.: 10/554,135

(22) PCT Filed: Apr. 19, 2004
(86) PCT No.: PCT/US04/12128

§ 371 Date: Sep. 26, 2006

(30) Foreign Application Priority Data
Apr. 25, 2003 (US) 60465718

(51) Int. Cl. C07D 207/16; C07C 201/12

(52) U.S. Cl. 548/531; 560/20

(57) ABSTRACT

A method of synthesizing a chiral compound having a quaternary carbon atom bearing diastereotopic groups from (a) a nitroolefin and (b) an α-substituted β-dicarbonyl or an equivalent compound having an acidic C—H moiety compound is disclosed. A subsequent intramolecular reaction between one of the substituents comprising the stereogenic carbon atom and one of the diastereotopic groups comprising the quaternary carbon atom creates a new compound having two contiguous stereogenic centers, one of which is quaternary, with control over the relative stereochemistry.

### FIELD OF THE INVENTION

The present invention relates to a method of preparing a chiral compound having a stereogenic carbon atom adjacent to a nonstereogenic quaternary carbon atom bearing diastereotopic groups. A subsequent intramolecular reaction between one of the substituents comprising the stereogenic carbon atom and one of the diastereotopic groups comprising the quaternary carbon atom creates a new compound containing two contiguous stereogenic centers, one of which is quaternary, with control over the relative and absolute stereochemistry.

### BACKGROUND OF THE INVENTION

Many organic compounds exist in optically active forms, i.e., they have the ability to rotate the plane of plane-polarized light. The different optically active forms of a compound are termed stereoisomers. A specific stereoisomer also can be referred to as an enantiomer, and a mixture of such stereoisomers often is called an enantiomeric, or racemic, mixture. For a given chemical compound, each of a pair of enantiomers are identical except that they are nonsuperimposable mirror images of one another.

Stereochemical purity is important in the pharmaceutical field, where many of the most often prescribed drugs exhibit chirality. For example, the L-enantiomer of the β-adrenergic blocking agent, propranolol, is known to be 100 times more potent than its D-enantiomer. Additionally, optical purity is important in the pharmaceutical drug field because certain stereoisomers impart a deleterious effect, rather than an advantageous or inert effect. For example, it is believed that the D-enantiomer of thalidomide is a safe and effective sedative when prescribed for the control of morning sickness during pregnancy, whereas its corresponding L-enantiomer is believed to be a potent teratogen.

Therefore, compounds that exhibit biological activity may contain one or more asymmetric carbon atoms. However, as stated above, one enantiomer of such a compound may exhibit excellent biological activity, whereas the other enantiomer may exhibit little biological activity, or may produce an undesired result. Accordingly, investigators strive to synthesize the biologically active enantiomer, while minimizing or eliminating synthesis of the inactive enantiomer.

The ability to selectively synthesize the desired enantiomer permits the preparation of a more useful drug product. For example, the administered dose of a drug can be reduced because only the active enantiomer is administered to an individual, as opposed to a racemic mixture which contains a large amount of the inactive enantiomer. This reduced dose of active enantiomer also reduces adverse side effects compared to a dose of the racemic mixture. In addition, a stereoselective synthesis is more economical because a step of separating the active and inactive enantiomers is eliminated, and raw material wastes and costs are decreased because raw materials are not consumed in the synthesis of the inactive enantiomer.

A particularly difficult problem encountered in the synthesis of a biologically active compound is the preparation of a quaternary carbon atom having a desired stereochemistry. A “quaternary carbon” is defined as a carbon atom having four substituents other than hydrogen. A quaternary carbon atom is asymmetric when the four substituents each are different from one another. Numerous synthetic reactions are available to form carbon-carbon bonds, but the number of available reactions to generate a quaternary carbon is limited. Furthermore, the number of readily available compounds having a tertiary carbon (defined as a carbon atom having one hydrogen atom and three substituents that are not hydrogen) as a starting material to generate an asymmetric quaternary carbon are limited. The stereoselective preparation of a quaternary carbon is even more challenging, and is an active area of research.

Typically, the formation of a quaternary carbon atom is a multistep process. In addition, reactions used to form quaternary carbon atoms often lead to unwanted side reactions. For example, reaction of a tertiary alkyl halide with an enolate leads to extensive elimination by dehydrohalogenation rather than substitution. Some of the difficulties in preparing a quaternary carbon atom are disclosed in WO 00/15599; S. F. Martin, Tetrahedron, 36, pages 419-460 (1980); K. Fuji, Chem. Rev., 93, pages 2037-2066 (1993); and E. J. Corey et al., Angew. Chem. Int. Ed., 37, pages 388-401 (1998).

### SUMMARY OF THE INVENTION

The present invention relates to a method of preparing a compound having a stereogenic carbon atom adjacent to a nonstereogenic carbon atom having diastereotopic groups. More particularly, the present invention is directed to a method of preparing a chiral compound having a stereogenic carbon atom of desired stereochemistry adjacent to a stereogenic quaternary carbon atom of desired stereochemistry by (a) reacting a nitroolefin with an α-substituted β-dicarbonyl compound or an equivalent compound having an acidic C—H moiety, (b) subsequent reduction of the nitro group, (c) followed by intramolecular cyclization onto a substituent, and typically a carbonyl substituent, of the prochiral center at the quaternary carbon atom to provide a cyclic compound containing two adjacent stereogenic carbon atoms, one of which is quaternary, with control over the relative and absolute stereochemistry.

Prior investigators attempted to prepare a ring system containing a quaternary carbon atom of desired stereochemistry by performing a cyclization and alkylation sequence to generate the quaternary carbon atom. These attempts led to racemic mixtures and side reactions that adversely affected reaction yield. The present method prepares chiral, and typically prochiral, quaternary carbon atoms prior to cyclization. A subsequent reduction and cyclization sequence provides a ring compound wherein a quaternary carbon atom of desired stereochemistry is positioned in a ring system adjacent to a chiral carbon of desired stereochemistry generated during a 1,3-dicarbonyl, or equivalent, addition.

More particularly, the present invention is directed to a method of preparing a compound having a stereogenic carbon atom of desired stereochemistry adjacent to a nonstereogenic quaternary carbon atom bearing diastereotopic groups by an addition reaction between a compound having a structural formula (I), and preferably a structural formula (Ia), and a nitroolefin (II) to yield a nitro compound (III), mediated by a catalyst complex comprising a ligand and a metal complex. The enantioselectivity of the addition is controlled by reaction conditions.

In one embodiment, the nitro (NO₂) 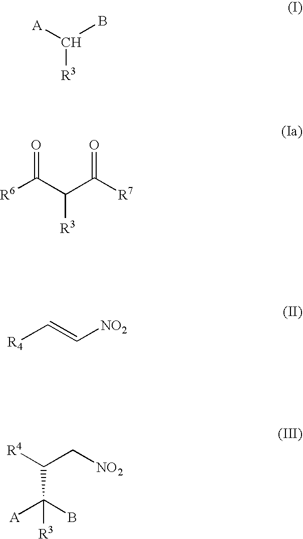
group of compound (III), or its enantiomer, is converted to an amino (NH₂) group to yield compound (IV), which then is subjected to an intramolecular cyclization reaction to yield compound (V) having a quaternary carbon of desired stereochemistry positioned in a ring system adjacent to the chiral carbon generated in the addition of the α-substituted β-dicarbonyl, or equivalent, compound to the nitroolefin. The diastereoselectivity of the cyclization is controlled by reaction conditions, and particularly, the temperature of the reaction. Most commonly, the cyclization is mediated by use of an amine or organometallic base.

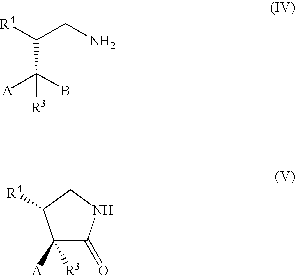

Therefore, an important aspect of the present invention is to provide a method of stereoselectively producing a nitro compound (III) from a nitroolefin (II) and a compound of structural formula (I), and particularly (Ia); wherein A is selected from the group consisting of C(═O)OR¹, C(═O)N(R⁵)₂, C(═O)SR⁵, CN, NO₂, and SO₂R⁵; B is selected from the group consisting of C(═O)OR², C(═O)N(R⁵)₂, C(═O)SR⁵, and CN; R¹ is selected from the group consisting of C₁₋₄alkyl, hydro, and M; R² is selected from the group consisting of hydro, M, alkoxyalkyl, alkyl, cycloalkyl, aryl, C₁₋₃alkylenearyl, heteroaryl, and C₁₋₃alkyleneheteroaryl; R³ is selected from the group consisting of C₁₋₄alkyl, alkoxy, acylamino, halos alkylthio, allyl, C₁₋₃alkylenearyl, and cyanoC₁₋₃alkyl; R₄ is selected from the group consisting of unsubstituted or substituted aryl and heteroaryl; R⁵, independently, is selected from the group consisting of hydro, C₁₋₄alkyl, cycloalkyl, aryl, C₁₋₃alkylenearyl, heteroaryl, and C₁₋₃alkyleneheteroaryl; and M is an alkali metal cation or an alkaline earth metal cation; and wherein R⁶ is alkoxy, amino, or thio; and R⁷ is selected from the group consisting of alkoxy, alkoxyalkyl, alkyl, cycloalkyl, aryl, C₁₋₃alkylenearyl, heteroaryl, and C₁₋₃alkyleneheteroaryl, in the presence of a catalyst complex and base, which generates a quaternary carbon adjacent to a chiral tertiary carbon. In preferred embodiments of compound (1a), R⁶ and R⁷ are the same alkoxy, which generates a quaternary carbon atom bearing two diastereotopic groups adjacent to a chiral tertiary carbon. In each case, R³ is selected from the group consisting of C₁₋₄alkyl, alkoxy, alkylthio, C₁₋₃alkylenearyl (e.g., benzyl), acylamino, halo, allyl, and cyanoC₁₋₃alkyl; and R⁴ is selected from the group consisting of unsubstituted or substituted aryl and heteroaryl. For R⁴, an electron-withdrawing substituent or an electron-donating aromatic group may be selected. Typically, electron-donating aromatic nitrostyrenes exhibit faster reaction times.

Other useful compounds of structural formula (I) include, but are not limited to:

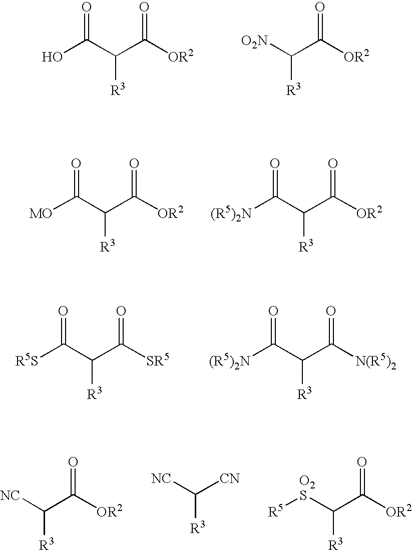

Examples of α-substituted β-diesters of structural formula (Ia) useful in the present invention include, but are not limited to:

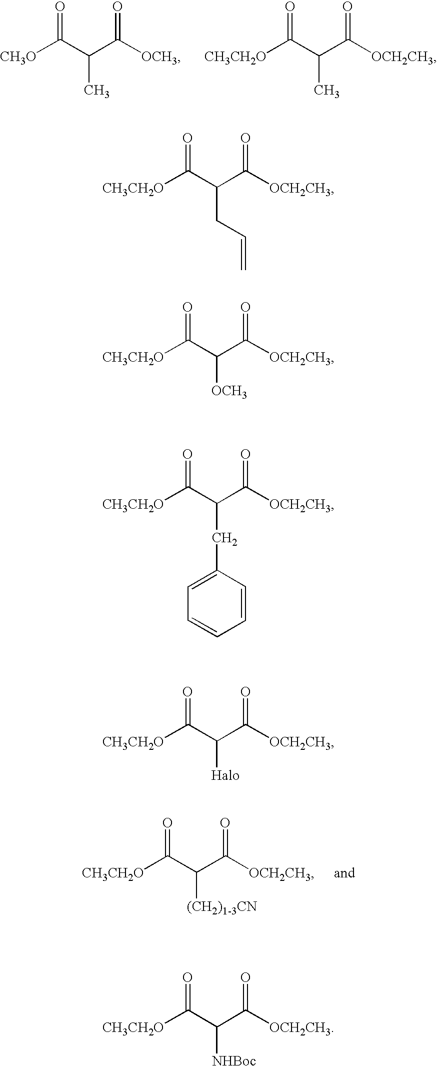

The catalyst complex comprises a ligand and a metal complex, wherein the ligand either has a structural formula (VI)

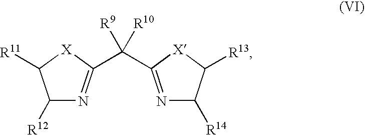

wherein R⁹ and R¹⁰, independently, are selected from the group consisting of hydro, alkyl, aryl, and C₁₋₃alkylenearyl, or R⁹ and R¹⁰ are taken together to form a 3-, 4-, 5-, or 6-membered cycloalkyl ring or a bicyclic ring;

X and X′, independently, are selected from the group consisting of oxygen, sulfur, and nitrogen;

R¹¹ and R¹², independently, are selected from the group consisting of hydro, alkyl, C₁₋₃alkylenearyl, and aryl, or R¹¹ and R¹² are taken together with the ring to which they are attached to form a bicyclic or tricyclic fused ring; and

R¹³ or R¹⁴, independently, are selected from the group consisting of hydro, alkyl, C₁₋₃alkylenearyl, and aryl, or R¹³ and R¹⁴ are taken together with the ring to which they are attached to form a bicyclic or tricyclic fused ring;

or has a structural formula (VII)

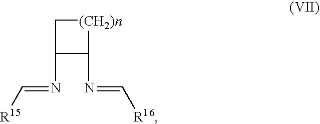

wherein n is 1-3, and R¹⁵ and R¹⁶, independently, are selected from the group consisting of alkyl, aryl, and C₁₋₃alkylenearyl. These ligands can be prepared in either chiral form and in high enantiomeric purity.

Another preferred ligand has a structural formula (XIII). or its enantiomer,

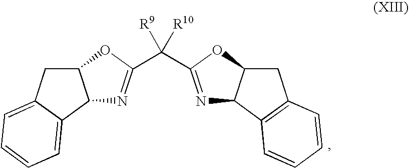

wherein R⁹ and R¹⁰, independently, are selected from the group consisting of methyl, ethyl, propyl, isopropyl, and C₁₋₃alkylenearyl, or R⁹ and R¹⁰ are taken together to form cyclopropyl, cyclobutyl, cyclopentyl, or indanyl.

Another aspect of the present invention is to provide an efficient racemic addition of a compound of structural formula (I), and preferably (Ia), to a nitroolefin. The use of racemic ligand (VI) or (VII) provides an efficient method of synthesizing racemic compounds. Previous attempts to achieve a racemic addition of α-substituted malonate diesters to nitrostyrenes required the use of the hazardous bases, like sodium metal and sodium hydride, and produced yields no greater than 65%. See B. Reichert et al., Chem. Ber., 71, 1254-1259 (1983); and N. Arai et al., Bull. Chem. Soc. Jpn., 70, 2525-2534 (1997). Attempts to repeat these methods using amine bases induced polymerization of the nitrostyrene. The use of a racemic mixture of ligands under the conditions disclosed herein provides the desired racemic addition product in high yield, while avoiding the use of hazardous bases.

A further aspect of the present invention relates to compounds prepared by the disclosed methods. In particular, the invention includes chiral compounds, as described herein, having a stereogenic carbon atom adjacent to a nonstereogenic quaternary carbon atom bearing diastereotopic groups, which are produced by the present methods.

These and other aspects and novel features of the present invention will become apparent from the following detailed description of the preferred embodiments.

### DETAILED DESCRIPTION OF THE PREFERRED EMBODIMENTS

The present invention is directed to a method of enantioselectively producing a nitro compound (III) from a nitroolefin (II) and a compound of structural formula (I), and preferably of structural formula (Ia), in the presence of a base and a catalyst complex comprising a chiral ligand and a metal complex, which generates a chiral or prochiral quaternary carbon adjacent to a chiral tertiary carbon.

More particularly, the present invention is directed to a method of preparing a compound having a quaternary carbon atom of desired stereoselectivity comprising reacting a compound having a structural formula (I) or (Ia)

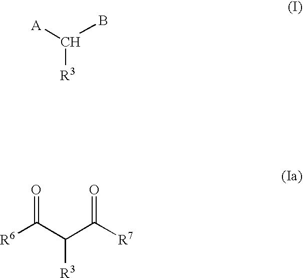

with a nitroolefin of structural formula (II)

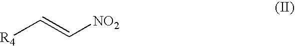

to form a nitro compound of structural formula (III) or (IIIa), respectively, or enantiomers thereof

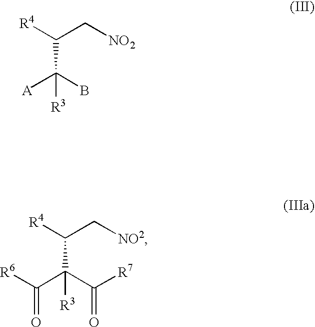

wherein A is selected from the group consisting of C(═O)OR¹, C(═O)N(R⁵)₂, C(═O)SR⁵, CN, NO₂, and SO₂R⁵; B is selected from the group consisting of C(═O)OR², C(═O)N(R⁵)₂, C(═O)SR⁵, and CN; R¹ is selected from the group consisting of C₁₋₄alkyl, hydro, and M; R² is selected from the group consisting of hydro, M, alkoxyalkyl, alkyl, cycloalkyl, aryl, C₁₋₃alkylenearyl, heteroaryl, and C₁₋₃alkyleneheteroaryl; R³ is selected from the group consisting of C₁₋₄alkyl, alkoxy, acylamino, halo, alkylthio, allyl, C₁₋₃alkylenearyl, and cyanoC₁₋₃alkyl; R₄ is selected from the group consisting of unsubstituted or substituted aryl and heteroaryl; R⁵, independently, is selected from the group consisting of hydro, C₁₋₄alkyl, cycloalkyl, aryl, C₁₋₃alkylenearyl, heteroaryl, and C₁₋₃alkyleneheteroaryl; and M is an alkali metal cation or an alkaline earth metal cation;

and wherein R⁶ is alkoxy; and R⁷ is selected from the group consisting of alkoxy, alkoxyalkyl, alkyl, cycloalkyl, aryl, C₁₋₃alkylenearyl, heteroaryl, and C₁₋₃alkyleneheteroaryl,

said reaction performed in the presence of a base and a catalyst complex comprising a ligand and a metal complex.

In certain preferred embodiments, R⁶ and R⁷ of structural formula (Ia) are the same alkoxy, which generates a prochiral quaternary carbon adjacent to a chiral tertiary carbon. For each of these cases, R³ is selected from the group consisting of C₁₋₄alkyl, alkoxy, alkylthio, acylamino, halo, allyl, C₁₋₃alkylenearyl, and cyanoC₁₋₃alkyl; and R⁴ is selected from the group consisting of aryl and heteroaryl.

The catalyst complex comprises a ligand and a metal complex. The ligand either has a structural formula (VI)

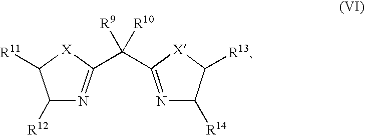

wherein R⁹ and R¹⁰, independently, are selected from the group consisting of hydro, alkyl, aryl, and C₁₋₃alkylenearyl, or R⁹ and R¹⁰ are taken together to form a 3-, 4-, 5-, or 6-membered cycloalkyl ring or a bicyclic ring;

X and X′, independently, are selected from the group consisting of oxygen, sulfur, and nitrogen;

R¹¹ and R¹², independently, are selected from the group consisting of hydra, alkyl, C₁₋₃alkylenearyl, and aryl, or R¹¹ and R¹² are taken together with the ring to which they are attached to form a bicyclic or tricyclic fused ring;

and R¹³ or R¹⁴, independently, are selected from the group consisting of hydro, alkyl, C₁₋₃alkylenearyl, and aryl, or R¹³ or R¹⁴ are taken together with the ring to which they are attached to form a bicyclic or tricyclic fused ring; or has a structural formula (VII)

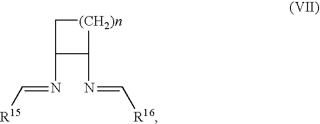

wherein n is 1-3, and R¹⁵ and R¹⁶, independently, are selected from the group consisting of alkyl, aryl, and C₁₋₃alkylenearyl.

In a preferred embodiment, R⁶ and R⁷ are alkoxy, R³ is selected from the group consisting of C₁₋₄ alkyl, alkoxy, acylamino, halogen, allyl, cyanomethyl, cyanoethyl and benzyl, and R⁴ is unsubstituted or substituted aryl or heteroaryl. In certain preferred embodiments, R⁶ and R⁷ are the same alkoxy, preferably methoxy or ethoxy. In other preferred embodiments, R⁴ is

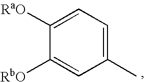

wherein R^{a} and R^{b}, independently, are selected from the group consisting of C₁₋₄alkyl, cycloalkyl, C₁₋₃alkyleneC₃₋₆cycloalkyl, heterocycloalkyl, C₁₋₃alkylenearyl, C₁₋₃alkyleneheteroaryl, aryl, and heteroaryl. In preferred embodiments, R^{a} and R^{b}, independently, are selected from the group consisting of methyl, benzyl, cyclopentyl, indanyl, cyclopropylmethyl, C₁₋₄alkylenephenyl, phenyl, substituted phenyl, thiazolyl, benzimidazolyl, tetrahydrofuryl, C₁₋₃alkylenethienyl, pyranyl, and C₁₋₃alkylenetetrafuryl. Several additional suitable R^{a} and R^{b} substituents are disclosed in U.S. Pat. No. 6,423,710, incorporated herein by reference. In especially preferred embodiments, R^{b} is C₁₋₄alkyl, particularly methyl.

The methods disclosed herein are useful in industrial applications, such as in the production of pharmaceuticals and agricultural chemicals. In particular, the methods disclosed herein are useful in synthesizing pharmaceuticals of high optical purity and having a heteroatom-containing ring system further containing a tertiary carbon atom of desired stereochemistry adjacent to a quaternary carbon atom of desired stereochemistry.

As used herein, the term “alkyl” is defined as straight chain and branched hydrocarbon groups containing the indicated number of carbon atoms. Unless otherwise indicated, the hydrocarbon group can contain up to 16 carbon atoms. Preferred alkyl groups are C₁₋₄alkyl groups, i.e., methyl, ethyl, and straight chain and branched propyl and butyl groups.

The term “cycloalkyl” is defined as a cyclic C₃-C₈ hydrocarbon group, e.g., cyclopropyl, cyclobutyl, cyclohexyl, and cyclopentyl. As defined herein, the term “cycloalkyl” includes “bridged alkyl,” i.e., a C₆-C₁₆ bicyclic or polycyclic hydrocarbon group, e.g., norbornyl, adamantyl, bicyclo-[2.2.2]octyl, bicyclo[2.2.1]heptyl, bicyclo[3.2.1]-octyl, and decahydronaphthyl. Cycloalkyl groups can be unsubstituted or substituted with one, two, or three substituents independently selected from the group consisting of C₁₋₄alkyl, haloalkyl, alkoxy, alkylthio, amino, alkylamino, dialkylamino, hydroxy, halo, mercapto, nitro, carboxaldehyde, carboxy, alkoxycarbonyl, and carboxamide.

The term “heterocycloalkyl” is defined herein as monocyclic, bicyclic, and tricyclic groups containing one or more heteroatoms selected from the group consisting of oxygen, nitrogen, and sulfur. A “heterocycloalkyl” group also can contain an oxo group (═O) attached to the ring. Nonlimiting examples of heterocycloalkyl groups include-1,3-dioxolanyl, 2-pyrazolinyl, pyrazolidinyl, pyrrolidinyl, piperazinyl, pyrrolinyl, 2H-pyranyl, 4H-pyranyl, morpholinyl, thiomorpholinyl, piperidinyl, 1,4-dithianyl, and 1,4-dioxanyl.

The term “alkylene” is defined herein as an alkyl group having a substituent. For example, the terms “C₁₋₃alkylenearyl” and “C₁₋₃alkeneheteroaryl” are defined as a C₁₋₃alkylene group substituted with an aryl or heteroaryl group, e.g., benzyl (—CH₂C₆H₅).

The term “halogen” is defined herein as fluorine, bromine, chlorine, and iodine. The term “halo” is defined herein as fluoro, bromo, chloro, and iodo.

The term “haloalkyl” is defined herein as an alkyl group substituted with one or more halo substituents. Similarly, “halocycloalkyl” is defined as a cycloalkyl group having one or more halo substituents.

The term “aryl,” alone or in combination, is defined herein as a monocyclic or polycyclic aromatic group, preferably a monocyclic or bicyclic aromatic group, e.g., phenyl or naphthyl. Unless otherwise indicated, an “aryl” group can be unsubstituted or substituted with one or more, and in particular one to three substituents, e.g., halo, alkyl, hydroxy, alkoxycarbonyl, carbamoyl, carboxy, carboxyaldehyde, hydroxyalkyl, alkoxy, alkoxyalkyl, haloalkyl, haloalkoxy, cyano, nitro, amino, alkylamino, acylamino, mercapto, alkylthio, alkylsulfinyl, and alkylsulfonyl. Examples of aryl groups include, but are not limited to, phenyl, naphthyl, tetrahydronaphthyl, chlorophenyl, methylphenyl, methoxyphenyl, trifluoromethylphenyl, nitrophenyl, and the like.

The term “heteroaryl” is defined herein as a monocyclic or bicyclic ring system containing one or two aromatic rings and containing at least one nitrogen, oxygen, or sulfur atom in an aromatic ring, and which can be unsubstituted or substituted with one or more, and in particular one to three, substituents, e.g., halo, alkyl, hydroxy, hydroxyalkyl, alkoxy, haloalkoxy, alkoxyalkyl, haloalkyl, perhaloalkyl, nitro, amino, alkylamino, acylamino, carbamoyl, carboxy, carboxyaldehyde, mercapto, alkylthio, alkylsulfinyl, and alkylsulfonyl. Examples of heteroaryl groups include, but are not limited to, thienyl, furyl, pyridyl, oxazolyl, quinolyl, isoquinolyl, indolyl, triazolyl, isothiazolyl, isoxazolyl, imidazolyl, benzothiazolyl, pyrazinyl, pyrimidinyl, thiazolyl, and thiadiazolyl.

The term “hydroxy” is defined herein as —OH.

The term “alkoxy” is defined herein as —OR, wherein R is alkyl, preferably C₁₋₄alkyl. The term “haloalkoxy” is defined herein as —OR, preferably C₁₋₄alkyl, wherein R is halo-substituted alkyl.

The term “alkoxyalkyl” is defined herein as an alkyl group wherein a hydrogen has been replaced by an alkoxy group. The term “(alkylthio)alkyl” is defined similarly as alkoxyalkyl, except that a sulfur atom is substituted for the oxygen atom.

The term “hydroxyalkyl” is defined herein as a hydroxy group appended to an alkyl group.

The term “amino” is defined herein as NH₂, and the term “alkylamino” is defined herein as NR₂, wherein at least one R is alkyl and the second R is alkyl or hydro.

The term “acylamino” is defined herein as R^{a}C(═O)N(R^{b})—, wherein R^{a} is alkyl or aryl and —R^{b} is hydrogen, alkyl or aryl.

The term “carboxaldehyde” is defined herein as —CHO.

The term “carboxy” is defined herein as —COOH.

The term “alkoxycarbonyl” is defined herein as —C(═O)OR, wherein R is alkyl.

The term “carboxamide” is defined herein as —C(═O)N(R)₂, wherein each R, independently, is hydro or alkyl.

The term “mercapto” is defined herein as —SH.

The term “alkylthio” is defined herein as —SR, wherein R is alkyl.

The term “alkylsulfinyl” is defined herein as R—SO₂—, wherein R is alkyl.

The term “alkylsulfonyl” is defined herein as R—SO₃—, wherein R is alkyl.

The term “nitro” is defined herein as NO₂.

The term “cyano” is defined herein as —CN.

The term “allyl” is defined as —CH₂CH═CH₂.

The term “cyanoC₁₋₃alkyl” is defined as —CH₂CN, —C₂H₅—CN, and —C₃H₇CN.

The term “alkali metal cation” is defined as a lithium, sodium, potassium, or cesium ion.

The term “alkaline earth metal cation” is defined as a magnesium, calcium, strontium, or barium ion.

Where no substituent is indicated as attached to a carbon or a nitrogen atom, it is understood that the carbon atom contains the appropriate number of hydrogen atoms. As used herein, “Me” is methyl, “Et” is ethyl, “Bn” is benzyl, “Bu” is butyl, “Boc” is t-butoxycarbonyl, and “Ac” is acetyl (CH₃C—O).

Useful compounds of structural formula (I) include, but are not limited to: 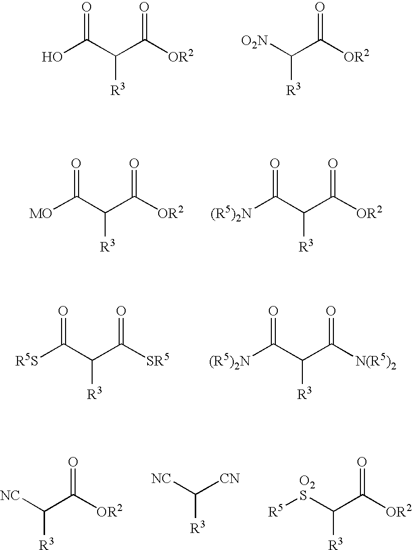
Examples of M include, but are not limited to, Na, K, Li, Mg, and Ca cations.

Examples of α-substituted β-diesters of structural formula (Ia) useful in the present invention include, but are not limited to:

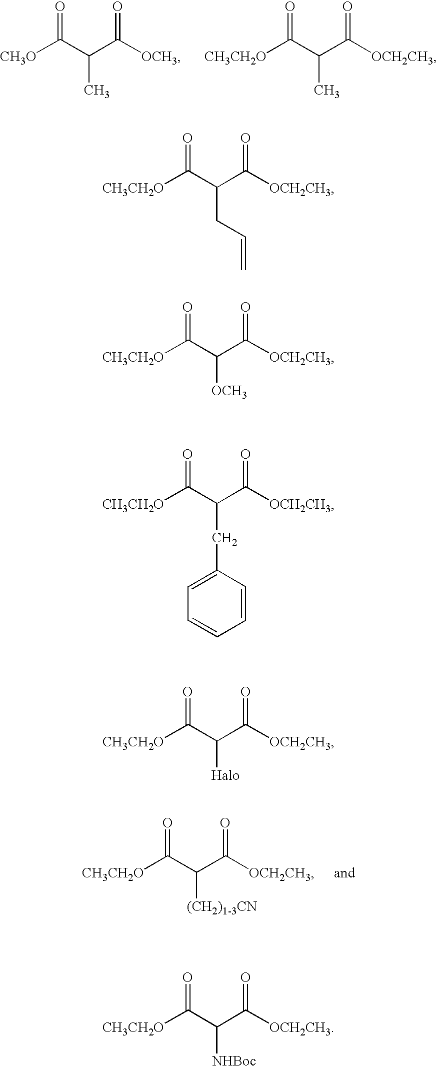

The addition reaction between a compound of structural formula (I), and particularly an α-substituted β-dicarbonyl compound (1a), and a nitroolefin (II) to form a nitro compound (III) is performed in the presence of a catalyst complex. The catalyst complex is formed by reacting a ligand and a metal complex. The ligand and the metal complex can be reacted in the presence of a solvent. The reaction time needed to form a catalyst complex is related to the identity of the ligand and the metal complex. Solvents useful in the formation of the catalyst complex include, but are not limited to, tetrahydrofuran (THF), toluene, methylene chloride (CH₂Cl₂), chlorobenzene, and chloroform (CHCl₃). Preferred solvents include chloroform and chlorobenzene.

Ligands useful in the preparation of the catalyst complex have a structural formula (VI) or (VII), such as are disclosed in WO 00/15599, and Johnson et al., Acc. Chem. Res., 33, 325-335 (2000), each incorporated herein by reference. Preferred ligands have a structural formula (VIII) or (IX)

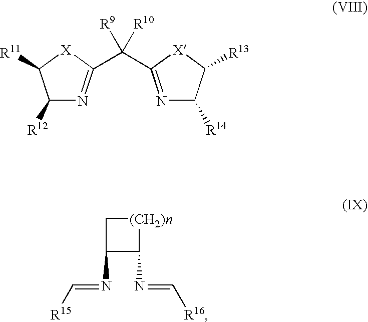

wherein n, X, X′, R⁹, R¹⁰, R¹¹, R¹²R¹³R¹⁴R¹⁵, and R¹⁶ are as defined above. Also preferred are enantiomers of compounds (VIII) and (IX).

A more preferred ligand has a structural formula (X)

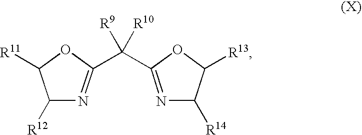

wherein R⁹ and R¹⁰, independently, are selected from the group consisting of methyl, ethyl, propyl, isopropyl, and C₁₋₃alkylenearyl, or R⁹ and R¹⁰ are taken together to form cyclopropyl, cyclobutyl, cyclopentyl, or indanyl, and R¹¹, R¹², R¹³, and R¹⁴, independently, are selected from the group consisting of hydro, alkyl, aryl, and C₁₋₃alkylenearyl.

Another preferred ligand has a structural formula (XI)

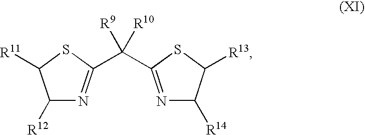

wherein R⁹ and R¹⁰, independently, are selected from the group consisting of methyl, ethyl, propyl, isopropyl, and C₁₋₃alkylenearyl, or R⁹ and R¹⁰ are taken together to form cyclopropyl; cyclobutyl, cyclopentyl, or indanyl, and R¹¹, R¹², R¹³, and R¹⁴ independently, are selected from the group consisting of hydro, alkyl, aryl, and C₁₋₃alkylenearyl.

Another preferred ligand has a structural formula (XIII)

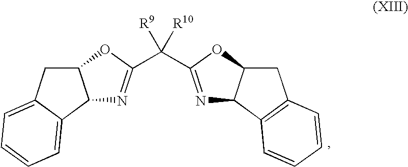

wherein R⁹ and R¹⁰, independently, are selected from the group consisting of methyl, ethyl, propyl, isopropyl, or C₁₋₃alkylenearyl, or R⁹ and R¹⁰ are taken together to form cyclopropyl, cyclobutyl, cyclopentyl, or indanyl, or the enantiomer of compound (XIII).

Metal complexes useful in the preparation of a catalyst complex include, but are not limited to, tin, zinc, aluminum, iron, nickel, titanium, ytterbium, zirconium, copper, antimony, or magnesium perchlorate; magnesium, copper, zinc, lanthanum, or nickel trifluoromethanesulfonate; magnesium, copper, zinc, or nickel bromide; magnesium, copper, zinc, or nickel iodide; magnesium, copper, zinc, or nickel acetylacetonate. A preferred metal complex is magnesium trifluoromethanesulfonate (Mg(OTf)₂).

A base useful in the reaction is an amine, preferably a tertiary amine. Suitable bases include, but are not limited to, triethylamine, diisopropylethylamine, 2,6-lutidine, N-methylmorpholine, N-ethylpiperidine, imidazole, and 5,6-dimethylbenzimidazole. The preferred bases are 2,6-lutidine, N-methylmorpholine, and 5,6-dimethylbenzimidazole. Use of stronger bases may result in polymerization of the nitrostyrene.

The stereoselectivity of the synthesis of nitro compound (III) can be controlled by the amount of catalyst complex used in the reaction and the time of reaction. In general, the addition of greater than about 5 mol % of the catalyst complex to the reaction mixture can result in high conversions after about a three-hour reaction time, however the stereoselectivity may not be fully optimized. To increase the stereoselectivity of the reaction, it has been useful in certain situations to use about 0.01 mol % to about 2 mol % catalyst, preferably about 0.05 mol % to about 1 mol %, e.g., about 0.1 mol % catalyst, and to extend reaction times to about 16 to about 30 hours, and preferably about 18 to about 24 hours. If the reaction proceeds for longer than about 30 hours, the enantiomeric excess of the product may decrease. A decreased in enantiomeric excess is more pronounced for methyl esters of α-substituted-β-dicarbonyl compounds (1a) than for ethyl esters, while isopropyl esters exhibit little or no decrease in enantiomeric excess.

The amount of base used in the reaction typically is slightly greater than the amount of catalyst complex, and is at least equal to the amount of catalyst complex. For example, when 1 mol % catalyst complex is used in the reaction, the amount of base typically is about 1 to about 7 mol %, preferably about 4 to about 6 mol %.

Cyclization of the nitro compound (III) is achieved using a two-step process, i.e., reduction of the nitro group followed by cyclization (lactamization), to yield the pyrrolidinone (V) containing two contiguous stereocenters. The level of stereoselectivity at the quaternary carbon atom of compound (V) is influenced by the identity of the chiral center of compound (III), as well as the steric bulk of the A and B groups and the conditions of the cyclization reaction.

Reduction of the nitro group can be performed by methods known in the art, preferably by reduction with nickel borohydride (prepared in situ from NiCl₂/NaBH₄, preferred mole ratio of <1:2.5), or by zinc reduction in the presence of an acid or by hydrogenation in the presence of a transition metal catalyst. If the nitro group is reduced to an amino group using zinc metal and an acid, the stereoselectivity of the reaction can be improved by removing any unreacted zinc prior to the cyclization step.

Cyclization proceeds in the presence of base and at a pH of about 9 or greater, e.g., about 9 to about 12, preferably about 9.5 to about 11. The temperature is not particularly critical, but a low temperature, preferably about −10° C. to about −78° C., more preferably, at about 20° C. to about −78° C., is used to improve diastereoselectivity. Nickel borohydride and Raney nickel reactions typically are performed at about 20° C. to about 70° C.

Suitable bases include organometallic bases, alkoxides, amines, and inorganic bases. Examples of specific bases include, but are not limited to, 1,8-diazabicyclo[5.4.0]undec-7-ene (DBU), sodium ethoxide (NaOEt), diisopropylethylamine, triethylamine, N-methylmorpholine, sodium bicarbonate, sodium carbonate, potassium hydroxide, sodium hydroxide, lithium hexamethyldisilazide, and isopropyl magnesium chloride. DBU is an especially preferred base.

A diethyl ester of compound (IV) (i.e., A and B are C(═O)OC₂H₅) appears to provide the greatest stereoselectivity. However, cyclization using a dimethyl ester of compound (IV) (i.e., A and B are C(═O)OCH₃) is still stereoselective, but the diastereomeric excess of the product may be reduced. When A and B are C(═O)OCH(CH₃)₂, a temperature greater than about −78° C. is needed for the cyclization reaction to proceed.

The R³ substituent of nitro compound (III) also influences the stereoselectivity of the cyclization reaction. As the R³ substituent increases in size, stereoselectivity of the cyclization reaction decreases. Therefore, preferred R³ substituents are methyl and ethyl.

### EXAMPLE 1

The following synthetic sequence illustrates the method of the present invention, wherein a stereogenic tertiary carbon is generated adjacent to a nonstereogenic quaternary carbon atom bearing diastereotopic groups by addition of an α-substituted malonate to a nitroolefin. Subsequent reduction of the nitro group to an amine group, followed by a stereoselective intramolecular cyclization of the amine compound produces a ring containing a chiral tertiary carbon atom adjacent to a chiral quaternary carbon atom. 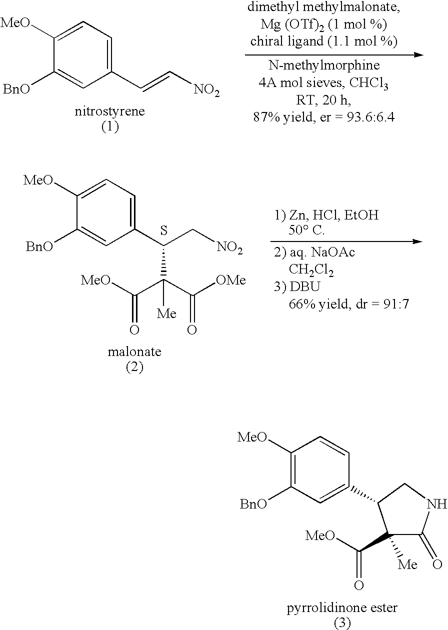
dimethyl methylmalonate, Mg(OTf)₂ (1 mol %) chiral ligand (1.1 mol %) N-methylmorphine 4A mol sieves, CHCl₃ RT, 20 h, 87% yield, er=93.6:6.4 nitrostyrene (1) malonate (2) 1) Zn, HCl, EtOH MeO 50° C. 2) aq. NaOAc BnO CH₂Cl₂ MeO Me 3) DBU 66% yield, dr=91:7 pyrrolidinone ester (3)

The chiral ligand used in the above synthetic sequence was:

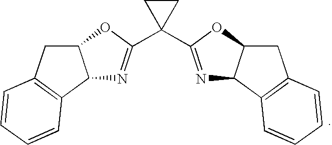

### Preparation of 2-Benzyloxy-1-methoxy-4-(2-nitrovinyl)benzene (nitrostyrene (1))

Nitrostyrene (1), also known as 3-benzyloxy-4-methoxy-β-nitrostyrene, was prepared from commercially available O-benzyl isovanillin (Aldrich Chem. Co., Milwaukee, Wis.) using the procedure disclosed in A. Bermejo et al., J. Med. Chem., 45, 5058-5086 (2002) or in Battersby, Tetrahedron, 14, 46-53 (1961).

### Preparation of 2-[(S)-1-(3-Benzyloxy-4-methoxyphenyl)-2-nitroethyl]-2-methylmalonic acid dimethyl ester (malonate (2))

Chloroform (4320 mL), the chiral ligand prepared as disclosed hereafter (54.8 g, 0.154 moles) and Mg(OTf)₂ (45.2 g, 0.14 moles) were added to a 50 L five-necked flask. The resulting mixture was stirred for at least 20 minutes, followed by adding water (10.4 mL), and stirring for at least one hour. Chloroform (11.48 L) and powdered 4 Å molecular sieves (784 g) were added to the reaction mixture, and stirring was continued for one hour, or until the water content was less than 40 ppm, as determined by Karl Fischer titration. Nitrogen gas (N₂) was bubbled through the reaction mixture for 0.5 hour, then nitrostyrene (1) (4 kg, 14.0 moles) was added as a solid over 20 minutes. Chloroform (250 mL) was added as a rinse, followed by the addition of dimethyl methylmalonate (2.482 kg, 16.96 moles, 2260.5 mL) over one minute. After rinsing with CHCl₃ (250 mL), N-methylmorpholine (18.4 g, 0.182 moles, 20 mL) was added rapidly via syringe. The reaction mixture was stirred under N₂ for 18 hours at room temperature (RT). The reaction was monitored for completion by HPLC. Then, water (1.6 L) was added to quench the reaction, followed by stirring at least one hour to allow the molecular sieves to swell. Next; the reaction mixture was filtered through a bed of CELITE™ on a coarse sintered glass funnel. The layers of the filtrate were separated, then the organic layer was washed with 1:1 brine:water solution (8 L). The organic layer was concentrated by rotary evaporation to provide a solid suspension. Ethanol (EtOH) (200 proof, 8 L) was added to the suspension, and the solids collected by filtration. The solid cake was washed with a minimum amount of 200 proof EtOH (500 mL). The wet cake then was added to a 50 L flask and triturated with EtOH (190 proof, 36 L) for 2 hours at 50° C., then allowed to cool to room temperature over 15 hours. The product was isolated by filtration, and the off-white crystalline solid dried under vacuum at 40-50° C. to give the desired product (2) (5.28 kg, 12.23 moles, 87% yield).

The purity of compound (2) by HPLC was 99%, and the enantiomeric ratio (e.r.) was 93.6:6.4. R_{f}=0.34 (2:1 hexane:EtOAc); ¹H NMR (CDCl₃/400 MHz) δ: 7.39 (br, d, 2H, Bn—H), 7.34 (br t, 2H, Bn—H), 6.78 (d, J=8.4 Hz, 1H, Ar—H), 6.68 (dd, J=2.0, 8.4 Hz, Ar—H), 6.66 (d, J=2.0 Hz, 1H, Ar—H), 5.13 (d, J=12.30, 1H, —OCH₂—Ar), 5.09 (d, J=12.30, 1H, —OCH₂—Ar), 4.91 (d, J=7.2 Hz, 2H, NO₂—CH₂), 4.00 (t, J=7.2 Hz, 1H, NO₂CH₂CHAr), 3.82 (s, 3H, Ar—OCH₃), 3.67 (s, 3H, —OCO₂CH₃), 3.65 (s, 3H, —CO₂CH₃), 1.21 (s, 3H, q. CH₃). ¹³C NMR (CDCl₃/400 MHz) δ: 171.53, 170.89, 149.94, 147.99, 136.98, 128.69, 128.03, 127.47, 127.16, 122.02, 115.69, 111.83, 77.75, 71.33, 56.97, 55.97, 53.12, 52.90, 48.10, 20.34. Rotation: [α]²⁴=+28.7 (c=1, chloroform). Anal. Calcd for C₂₂H₂₅NO₈: C, 61.25; H, 5.84; N, 3.25. Found: C, 61.11; H, 5.96; N, 3.15. RP-HPLC Conditions: Waters YMC-Pack Pro-C18, 120 Å, 5 μm, 4.6 mm×150 mm with mobile phases A; Water, 0.1% trifluoroacetic acid, 1% isopropyl alcohol; B: acetonitrile, 0.05% trifluoroacetic acid, 1% isopropyl alcohol at 1.5 mL/min using a gradient from 15% B to 95% B over 10 minutes, hold at 95% B for 2.5 minutes, return to 15% B in one minute, hold at 15% B for 1.5 minutes. UV detection at 233 nm t_{R}=9.7 min. Chiral HPLC conditions: CHIRALPAK® AD column, 10 μm, 4.6 mm×250 mm with hexane-ethanol (90:10, v/v) mobile phase at 1.0 mL/min. UV detection at 206 nm, t_{g}=11.4 min.

The chiral ligand used in the above reaction was prepared as follows. Also see I. W. Davies et al., Tet. Lett., 37, pp. 813-814 (1996) and Chem. Commun., pp. 1753-1754 (1996). 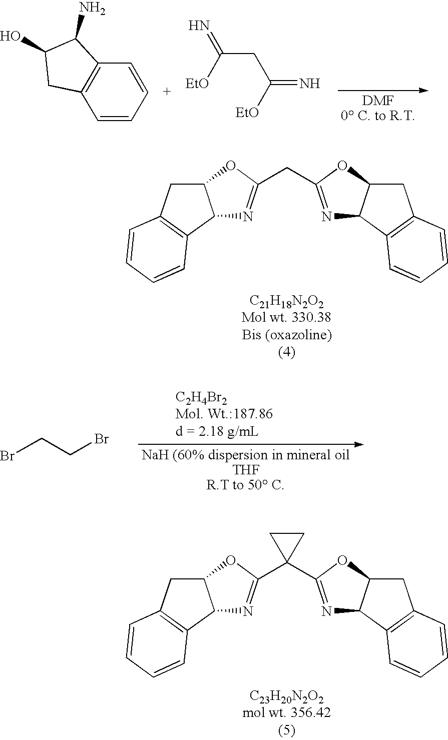
DMF 0° C. to R.T. C₂₁H₁₈N₂O₂ Mol wt. 330.38 Bis(oxazoline) (4) C₂H₄Br₂ Mol. Wt.: 187.86 d=2.18 g/mL NaH (60% dispersion in mineral oil) THF C₂₃H₂₀N₂O₂ mol wt. 356.42 (5)

### Preparation of [3aR-[2(3′aR*,8′aS*),3′aβ,8′aβ]]-(+)-2,2′-methylene bis-[3a,8a-dihydro-8H-indeno-[1,2-d]-oxazole (bis(oxazoline) (4))

A 3 L round bottom flask was charged with diethyl malonimidate dihydrochloride (25.8 g, 0.112 moles, 1.0 equiv.) and dimethylformamide (DMF) (320 mL). The mixture was cooled in an ice bath. To this suspension was added (1R,2S)-(+)-cis-1-amino-2-indanol (40 g, 0.268 moles, 2.4 equivalents), in portions, over twenty minutes. The ice bath then was removed, and the reaction allowed to warm to room temperature, during which time the reaction product precipitated from the reaction. After four days stirring at room temperature, the reaction was filtered. The collected white solid was suspended in CH₂Cl₂ (450 mL). The mixture then was washed with water (260 mL) and brine (260 mL). The organic layer was dried over sodium sulfate (Na₂SO₄), filtered, and concentrated to an off-white solid. Drying overnight under vacuum provided 23.9 g (65% yield) of the bis(oxazoline) (4). ¹H NMR (300 MHz/CDCl₃): δ 7.45 (m, 2H, Ar—H); 7.27-7.21 (m, 6H, Ar—H); 5.56 (d, J=7.9 Hz, 2H, N—CH); 5.34 (m, 2H, O—CH); 3.39 (dd, J=7.0, 18.0 Hz, 2H, Ar—CHH); 3.26 (s, 2H, —CH₂—); 3.16 (d, J=18.0 Hz, 2H, 14-CHH). The NMR is consistent with the peak assignments made in WO 00/15599.

### Preparation of [3aR-[2(3′aR*,8′aS*),3′aβ,8′aβ]]-(+)-2,2′-cyclopropylidene bis[3a, 8a-dihydro-8H-indeno-[1,2-d]oxazole (chiral ligand (5))

To a 1 L round bottom flask was added the bis(oxazoline) (4) (30.3 g, 91.7 mmole, 1 equiv.), and dry THF (450 mL). The slurry was cooled to 0° C., and 60% sodium hydride (NaH) in mineral oil (11.0 g, 275.1 mmole, 3 equiv.) was added cautiously with stirring. The mixture was warmed to room temperature, then 1,2-dibromoethane (11.85 mL, 138 mmol, 1.5 equiv.) was added over 15 minutes while maintaining the temperature between 25° C. and 30° C. The reaction was warmed slowly to 50° C., then stirred for 3 hours. The reaction was monitored by TLC (10% methanol/ethyl acetate, starting material R_{f}-0.3 (streaky), product R_{f}-0.45 (not as streaky as the starting material)). After completion, the reaction mixture was cooled to 0° C., and carefully quenched with saturated ammonium chloride (NH₄Cl) (150 mL). Water (150 mL) was added, and the product was extracted twice with CH₂Cl₂ (450 mL and 150 mL). The combined organic layers were dried over Na₂SO₄, filtered, and concentrated to provide an orange solid. The solid was triturated with hexanes (240 mL) at room temperature, filtered, and then washed with additional hexanes (91 mL) to yield compound (5) (32 g, 98%) as a white powder. ¹H NMR (300 MHz/CDCl₃): δ 7.45 (m, 2H, Ar—H); 7.27-7.19 (m, 6H, Ar—H), 5.52 (d, J=7.7 Hz, 2H, N—CH); 5.32 (m, 2H, O—CH); 3.39 (dd, J=7.0, 18.0 Hz, 2H, Ar—CHH), 3.20 (dd, J=1.8, 18.0 Hz, 2H, Ar—CHH); 1.36 (m, 2H, —CHH—CHH—); 1.27 (m, 2H, —CHH—CHH—).

### Preparation of 4-(3-benzyloxy-4-methoxyphenyl)-3-methyl-2-oxo-pyrrolidine-3-carboxylic acid method ester (3)

To a flask containing the malonate (2) (20.0 g, 46.4 mmoles, 1.00 eq.) was added 190 proof EtOH (200 mL). Next, concentrated hydrochloric acid (HCl) (100 mL, 1200-mmoles, 25.9 eq.) was cautiously added via an addition funnel. The addition was very exothermic, and the reaction temperature increased from 23° C. to 48° C. To this mixture, zinc dust (28.5 g, 436 mmoles, 9.4 eq.) was added portionwise to maintain a temperature of 45° C. to 52° C. The reaction was monitored by HPLC. When the reaction was judged complete (hydroxylamine completely reduced to amine), the gray suspension was cooled to 0° C., then saturated aqueous sodium acetate (NaOAc) (100 ml) was added to the reaction mixture. The unreacted zinc dust then was removed by filtration. The filtrate was concentrated to remove the EtOH, then diluted with CH₂Cl₂ (200 mL). The layers were separated and the aqueous layer was extracted with CH₂Cl₂ (50 mL). The combined organic layers were washed with saturated aqueous NaOAc (200 mL). The organic layer was dried over Na₂SO₄ and filtered. The organic solution then was cooled to −78° C., then DBU (30 mL, 201 mmol, 4.33 eq.) was added. The resulting solution was stirred at −78° C. for 1 hour, then warmed to room temperature. HPLC analysis showed a 5:1 ratio of diastereomers.

The reaction mixture was poured into 1N HCl (200 mL), then the layers were separated. The aqueous layer then was extracted CH₂Cl₂ (25 mL). The combined organic layers were washed with 1N HCl (100 mL), and the layers were separated. The resulting organic layer was dried over Na₂SO₄, filtered, and concentrated. The product was isolated by crystallizing from methyl t-butyl ether to give pyrrolidinone ester (3) (11.4 g, 66% yield), with a 91:7 ratio of desired diastereomer to undesired diastereomer.

The above synthetic sequence illustrates the manufacture of a cyclic compound having a quaternary carbon of desired stereochemistry positioned in a ring system adjacent to a chiral tertiary carbon of desired stereochemistry. The pyrrolidinone ester (3) is prepared in good yield and excellent optical purity. The pyrrolidinone ester (3) can be subjected to a variety of reactions to provide useful commercial products including pharmaceuticals, without affecting the stereochemistry of the quaternary or tertiary ring carbons.

The following synthetic sequence illustrates the use of diethyl allyl malonate in the present method to generate a pyrrolidinone ester containing two contiguous stereocenters, one of which is quaternary bearing an allyl substituent that can be readily subjected to a variety of reactions to provide useful commercial products including pharmaceuticals, without affecting the stereochemistry of the quaternary or tertiary ring carbons.

### EXAMPLE 2

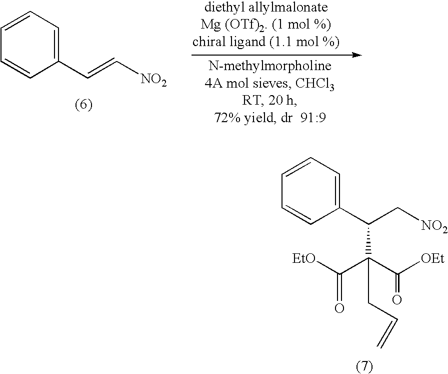 diethyl allylmalonate Mg(OTf)₂ (1 mol %) chiral ligand (1.1 mol %) N-methylmorpholine 4A mol sieves, CHCl₃RT, 20 h, (6) 72% yield, dr 91:9

The chiral ligand used in Example 2 was

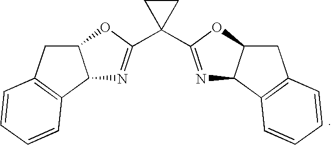

### Preparation of 2-[1R-phenyl-2-nitroethyl]-2-allylmalonic acid diethyl ester (7)

Chloroform (CHCl₃), or alternatively chlorobenzene, (2.5 mL), the chiral ligand (enantiomer) (34.25 mg, 0.097 mmoles), and Mg(OTf)₂ (28.25 mg, 0.088 mmoles) were added to a 25 mL flask. The resulting mixture was stirred for at least 20 minutes followed by the addition of water (0.0065 mL). The resulting mixture was stirred for at least 1 hour. The molecular sieves are an optional, but preferred, component, because stereoselectivity is improved when molecular sieves are present. Chloroform (7.5 mL) and powdered 4 Å molecular sieves (367.5 mg) were added to the reaction mixture, and stirring was continued for a minimum of 1 hour. Water content then was determined by Karl Fischer titration. If the water content was 40 ppm or greater, stirring was continued and additional molecular sieves were added. When the water content was less then 40 ppm, N₂ was bubbled through the reaction mixture for a minimum of 2 minutes. Nitrostyrene (6) (1.31 g, 8.77 mmoles) then was added as a solid over 1 minute. Chloroform (1 mL) was added as a rinse, followed by the addition of diethyl allylmalonate (2.13 g, 10.65 mmoles, 2.09 mL) over 1 minute via syringe. N-methylmorpholine (11.5 mg, 0.114 mmoles, 0.0125 mL) was added rapidly via pipette. Nitrogen gas was bubbled through the reaction mixture for a minimum of 2 minutes, and the reaction mixture then was stirred under nitrogen for 45 hours at RT. The reaction was monitored for completion by HPLC. Water (1 mL) was added to quench the reaction, and the reaction mixture was stirred at least 5 minutes to allow the molecular sieves to swell. Next, the reaction mixture was filtered through a bed of CELITE™. The layers of the filtrate were separated, then the organic layer was washed with brine (15 mL). The organic layer was dried over Na₂SO₄ (5 g). The organic layer was concentrated by rotary evaporation to provide a yellow oil. The oil was purified using flash chromatography by eluting with 9:1 hexanes:EtOAc. Chromatography was necessary to separate the starting material (R_{f}=0.4) and the product (R_{f}=0.31). After concentration under vacuum, the desired product (7) was obtained as a clear oil (2.2 g, 6.29 mmole, 72% yield). The purity by HPLC was >98 areas and the enantiomeric ratio was 91:9. R_{f}=0.31 (9:1 hexane:EtOAc). ¹H NMR (CDCl₃/400 MHz) δ: 7.32-7.27 (m, 3H, Ar—H), 7.14 (d, J=7.8 Hz, 1H, Ar—H), 7.13 (d, J=5.7 Hz, —H, Ar—H), 5.80-5.68 (m, 1H, CH═CH₂), 5.17-4.95 (m, 4H, CH═CH₂, CH₂—NO₂), 4.31 (q, J=7.14 Hz, 1H, —OCH₂Me), 4.30 (q, J=7.14 Hz, 1H, —OCH₂Me), 4.23 (q, J=7.14 Hz, 2H, —OCH₂Me), 4.19 (dd, J=3.07, 7.05 Hz, 1H, Ar—CH), 2.57 (dd, J=6.52, 14.51 Hz, 1H, C—CH₂), 2.27 (dd, J=8.01, 14.55 Hz, 1H, C—CH₂), 1.32 (t, J=7.08 Hz, 3H, —CH₃), 1.27 (t, J=7.08 Hz, 3H, —CH₃). ¹³C NMR (CDCl₃/400 MHz) δ: 169.92, 169.73, 135.26, 132.08, 129.15, 129.01, 128.67, 120.05, 78.77, 62.21, 60.67, 46.87, 38.60, 14.27. Rotation: [α]²⁴=−35.2 (c=1, chloroform). LCMS m/z 350 (M+1), 303, 275. Anal. Calcd for C₂₂H₂₅NO₈: C, 61.88; H, 6.64; N, 4.01. Found: C, 61.99; H, 6.97; N, 4.02.

### EXAMPLE 3

The above synthesis also can be performed using a racemic mixture of the ligand to generate a racemic mixture of a compound having a stereogenic carbon atom adjacent to a nonstereogenic carbon bearing diastereotopic groups. 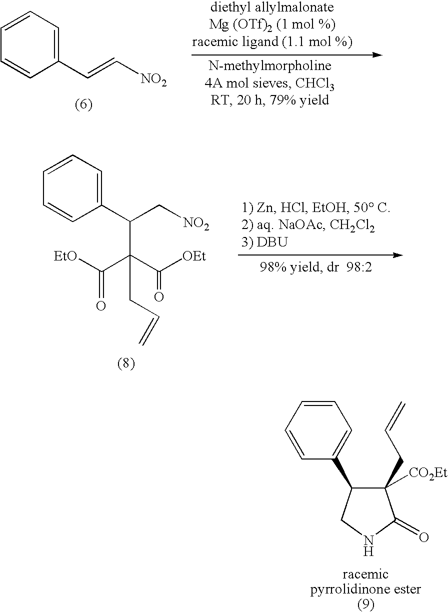
diethyl allylmalonate Mg(OTf)₂ (1 mol %) racemic ligand (1.1 mol %) N-methylmorpholine 4A mol sieves, CHCl₃ RT, 20 h, (6) 79% yield 1) Zn, HCl, EtOH, 50° C. 2) aq. NaOAc, CH₂Cl₂ 3) DBU 98% yield, dr 98:2 racemic pyrrolidinone ester (9)

### Preparation of 2-Allyl-2-[1-phenyl-2-nitroethyl]-malonic acid diethyl ester (8)

Chloroform (150 mL), racemic ligand (1.97 g, 5.52 mmoles), and Mg(OTf)₂ (1.62 g, 5.03 mmoles) were added to a 2 L flask. The mixture was stirred for at least 20 minutes followed by the addition of water (0.374 mL). The resulting mixture was stirred for at least 1 hour. Chloroform (450 mL) and powdered 4 Å molecular sieves (22.2 g) were added to the reaction mixture, and stirring was continued for a minimum of 1 hour. The water content then was determined by Karl Fischer titration. If the water content was 40 ppm or greater, stirring was continued and additional molecular sieves were added. When the water content was below 40 ppm, N₂ was bubbled through the reaction mixture for a minimum of 5 minutes. Nitrostyrene (6) (75 g, 502.9 mmoles) was added as a solid over 5 minutes. Chloroform (20 mL) was added as a rinse, followed by the addition of diethyl allylmalonate (110.76 g, 553.14 mmoles, 109.12 mL) over 2 minutes via graduated cylinder. N-methylmorpholine (661 mg, 6.54 mmoles, 0.719 mL) was added rapidly via pipette. Nitrogen gas again was bubbled through the reaction mixture for a minimum of 5 minutes. The reaction mixture was stirred under N₂ for 67 hours at room temperature. The reaction mixture was monitored for completion by HPLC. Water (50 mL) was added to quench the reaction, and the mixture was stirred at least 15 minutes to allow the molecular sieves to swell. Next, the reaction mixture was filtered through a bed of CELITE™. The layers of the filtrate were separated, then the organic layer was washed with 1:1 brine:water solution (375 mL). The organic layer was concentrated by rotary evaporation to provide over 200 g of a crude yellow oil. The oil was purified using a silica gel plug by eluting with a gradient starting at 20:1 and going to 9:1 hexanes:EtOAc. Chromatography was necessary to separate the starting materials (R_{f}=0.19, 20:1). After concentration under vacuum, a clear oil was obtained (124.3 g, 356 mmole, 71% yield). The purity of the product by HPLC was >97 area % and the product was a racemic mixture by HPLC. An additional 15.02 g was contained in an impure fraction as determined by wt % assay compared to an analytically pure standard. Therefore, the reaction gave a total of 132.32 g of compound (8) (399 mmole, 79% yield). R_{f}=0.19 (20:1 hexane:EtOAc). ¹H NMR (CDCl₃/400 MHz) δ: 7.32-7.27 (m, 3H, Ar—H), 7.14 (d, J=7.8 Hz, 1H, Ar—H), 7.13 (d, J=5.7 Hz, 1H, Ar—H), 5.80-5.68 (m, 1H, CH═CH₂), 5.17-4.95 (m, 4H, CH═CH₂, CH₂—NO₂), 4.31 (q, J=7.14 Hz, 1H, —OCH₂Me), 4.30 (q, J=7.14 Hz, 1H, —OCH₂Me), 4.23 (q, J=7.14 Hz, 2H, —OCH₂Me), 4.19 (dd, J=3.07, 7.05 Hz, 1H, Ar—CH), 2.57 (dd, J=6.52, 14.51 Hz, 1H, C—CH₂), 2.27 (dd, J=8.01, 14.55 Hz, 1H, C═CH₂), 1.32 (t, J=7.08 Hz, 3H, —CH₃), 1.27 (t, J=7.08 Hz, 3H, —CH₃).

### Preparation of 3-Allyl-2-oxo-4-phenyl-pyrrolidine-3-carboxylic acid ethyl ester (9)

To a flask containing compound (8) (120.0 g, 343.46 mmoles, 1.00 eq.) was added 190 proof EtOH (1500 mL). Next, concentrated HCl (710.7 mL, 8.65 moles, 25.2 eq.) was cautiously added via an addition funnel. The addition was very exothermic and the reaction temperature increased from 23° C. to 45° C. Zinc dust (211.1 g, 3.23 moles, 9.4 eq.) was added portionwise to maintain a temperature of 45° C. to 55° C. and monitored the reaction by HPLC. When the reaction was judged complete, the gray suspension was cooled to 0° C. The suspension was diluted with saturated aqueous-NaOAc (720 mL) at 0° C., and the unreacted zinc then was removed by filtration. The filtrate was concentrated to remove EtOH, then diluted with CH₂Cl₂ (1 L). The organic layer was washed with saturated aqueous NaOAc (300 mL), then dried over Na₂SO₄, and filtered. The organic solution was cooled to −78° C., then DBU (221 mL, 1.48 mol, 4.33 eq.) was added. The resulting solution was stirred at −78° C. for 1 hour, then warmed to room temperature. HPLC analysis showed a greater than 60:1 ratio of diastereomers. The reaction mixture then was poured into 1N HCl (400 mL) and the layers separated. The aqueous layer was extracted with CH₂Cl₂ (800 mL). The combined organic layers were washed with brine (500 mL), and the layers were separated. The organic layer was dried over Na₂SO₄, filtered, and concentrated. The product (9) was isolated as an oil, which crystallized upon sitting to give 92.07 g (98% yield), 98:2 ratio of desired diastereomer to undesired diastereomer. ¹H NMR (CDCl₃/400 MHz) δ: 7.33-7.25 (m, 3H, Ar—H), 7.20-7.15 (m, 2H, Ar—H), 6.74 (br s, 1H, N—H), 5.70-5.57 (m, 1H, CH═CH₂), 4.92 (d, J=10.5 Hz, 1H, CH═CH₂), 4.84 (dd, J=16.9, 3.13 Hz, 1H, CH═CH₂), 4.28 (q, J=7.13 Hz, 1H, —OCH₂Me), 4.27 (q, J=7.13 Hz, 1H, —OCH₂Me), 4.26 (t, J=6.83 Hz, 1H, Ar—CH), 3.75 (dd, J=7.12, 9.03 Hz, 1H, CH₂—NO₂), 3.61 (dd, J=6.35, 9.36 Hz, 1H, CH₂—NO₂), 2.41 (dd, J=7.76, 14.5 Hz, 1H, C—CH₂), 2.26 (dddd, J=1.46, 1.46, 6.68, 14.5 Hz, 1H, C—CH₂), 1.30 (t, J=7.25 Hz, 3H, —CH₃).

Compound (7) was subjected to similar conditions as above to yield a single diastereomer of chiral product (9) in 98% yield, 98:2 ratio of desired diastereomer to undesired diastereomer.

Obviously, many modifications and variations of the invention as hereinbefore set forth can be made without departing from the spirit and scope thereof, and, therefore, only such limitations should be imposed as are indicated by the appended claims.

1. A method of preparing a compound having a quaternary carbon atom of desired stereoselectivity comprising reacting a compound having a structural formula (I) 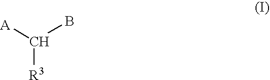
with a nitroolefin of structural formula (II) 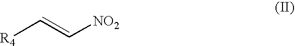
to form a nitro compound of structural formula (III) or its enantiomer 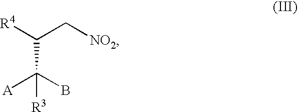
wherein A is selected from the group consisting of C(═O)OR¹, C(═O)N(R⁵)₂, C(═O)SR⁵, CN, NO₂, and SO₂R⁵; B is selected from the group consisting of C(═O)OR², C(═O)N(R⁵)₂, C(═O)SR⁵, and CN; R¹ is selected from the group consisting of C₁₋₄alkyl, hydro, and M; R² is selected from the group consisting of hydro, M, alkoxyalkyl, alkyl, cycloalkyl, aryl, C₁₋₃alkylenearyl, heteroaryl, and C₁₋₃alkyleneheteroaryl; R³ is selected from the group consisting of C₁₋₄alkyl, alkoxy, acylamino, halo, alkylthio, allyl, C₁₋₃alkylenearyl, and cyanoC₁₋₃alkyl; R₄ is selected from the group consisting of unsubstituted or substituted aryl and heteroaryl; R⁵, independently, is selected from the group consisting of hydro, C₁₋₄alkyl, cycloalkyl, aryl, C₁₋₃alkylenearyl, heteroaryl, and C₁₋₃alkyleneheteroaryl; and M is an alkali metal cation or an alkaline earth metal cation; and
said reaction performed in the presence of a base and a catalyst complex comprising a ligand and a metal complex.

2. A method of preparing a compound having a quaternary carbon atom of desired stereoselectivity comprising reacting an α-substituted β-dicarbonyl compound of structural formula (Ia) 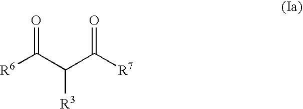
with a nitroolefin of structural formula (II) 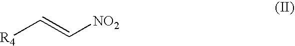
to form a nitro compound of structural formula (IIIa) or its enantiomer 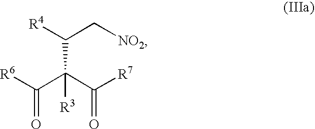
wherein R⁶ is alkoxy; R⁷ is selected from the group consisting of alkoxy, alkoxyalkyl, alkyl, cycloalkyl, aryl, C₁₋₃alkylenearyl, heteroaryl, C₁₋₃alkyleneheteroaryl; R³ is selected from the group consisting of C₁₋₄alkyl, alkoxy, acylamino, halo, alkylthio, allyl, C₁₋₃alkylenearyl, and cyanoC₁₋₃alkyl; and R⁴ is selected from the group consisting of unsubstituted or substituted aryl and heteroaryl;
said reaction performed in the presence of a base and a catalyst complex comprising a ligand and a metal complex.

3. The method of claim 1 or 2 wherein the ligand has a structural formula (VI) 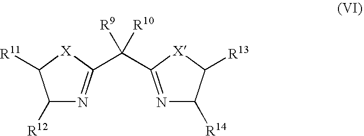
wherein R⁹ and R¹⁰, independently, are selected from the group consisting of hydro, alkyl, aryl, and C₁₋₃alkylenearyl, or R⁹ and R¹⁰ are taken together to form a 3-, 4-, 5-, or 6-membered cycloalkyl ring or a bicyclic ring;
X and X′, independently, are selected from the group consisting of oxygen, sulfur, and nitrogen;
R¹¹ and R¹², independently, are selected from the group consisting of hydro, alkyl, C₁₋₃alkylenearyl, and aryl, or R¹¹ and R¹² are taken together with the ring to which they are attached to form a bicyclic or tricyclic fused ring; and
R¹³ or R¹⁴, independently, are selected from the group consisting of hydro, alkyl, C₁₋₃alkylenearyl, and aryl, or R¹³ and R¹⁴ are taken together with the ring to which they are attached to form a bicyclic or tricyclic fused ring;
or has a structural formula (VII), 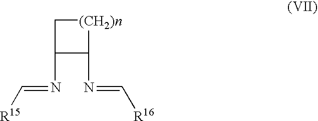
wherein n is 1-3, and R¹⁵ and R¹⁶, independently, are selected from the group consisting of alkyl, aryl, and C₁₋₃alkylenearyl.

4. The method of claim 1 or 2 wherein the metal complex is selected from the group consisting of magnesium perchlorate, magnesium trifluoromethanesulfonate, copper trifluoromethanesulfonate, zinc trifluoromethanesulfonate, lanthanum trifluoromethanesulfonate, nickel trifluoromethanesulfonate, magnesium bromide, copper bromide, zinc promide, nickel bromide, magnesium iodide, copper iodide, zinc iodide, nickel iodide, magnesium acetylacetonate, copper acetylacetonate, zinc acetylacetonate, nickel acetylacetonate, and mixtures thereof.

5. The method of claim 4 wherein the metal complex comprises magnesium trifluoromethanesulfonate.

6. The method of claim 1 or 2 wherein the base is selected from the group consisting of triethylamine, diisopropylethylamine, 2,6-lutidine, N-methylmorpholine, N-ethylpiperidine, imidiazole, and 5,6-dimethylbenzimidazole.

7. The method of claim 1 or 2 wherein the ligand has a structure 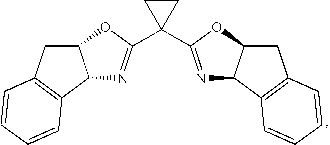
or its enantiomer.

8. The method of claim 2 wherein R⁶ and R⁷ are alkoxy.

9. The method of claim 8 wherein R⁶ and R⁷, independently, are methoxy or ethoxy, and R³ is methyl or ethyl.

10. The method of claim 1 wherein the compound of structural formula (I) has a structural formula

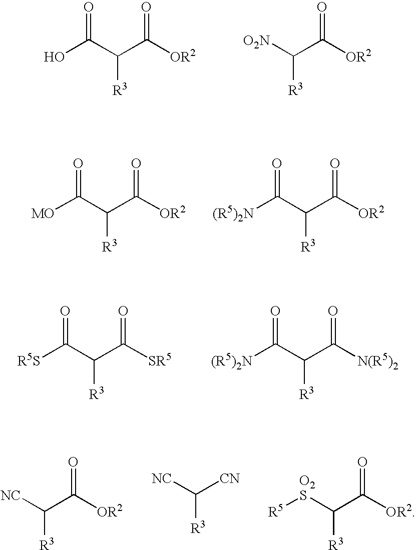

11. The method of claim 2 wherein the α-substituted β-carbonyl compound has a structural formula:

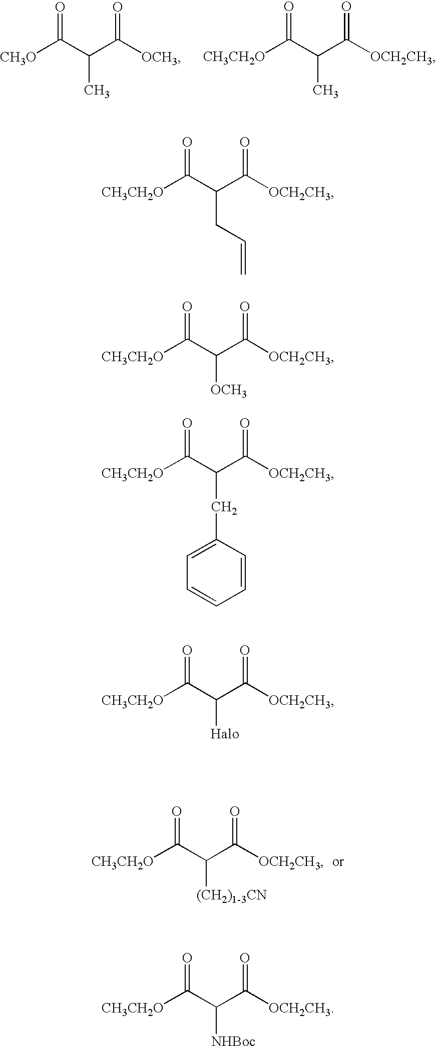

12. The method of claim 1 or 2 wherein R⁴ is aryl.

13. The method of claim 12 wherein R⁴ is substituted phenyl.

14. The method of claim 1 or 2 wherein R⁴ is 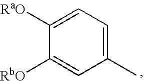
wherein R^{a} and R^{b}, independently, are selected from the group consisting of C₁₋₄alkyl, cycloalkyl, heterocycloalkyl, aryl, heteroaryl, C₁₋₃alkylenearyl, and heteroC₁₋₃alkylenearyl.

15. The method of claim 1 further comprising the steps of converting the nitro group of nitro compound (III) to form an amino compound (IV) 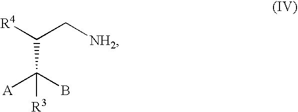
followed by an intramolecular cyclization reaction to form a compound (V)

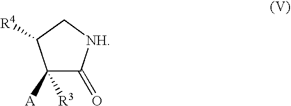

16. The method of claim 2 further comprising the steps of converting the nitro group of nitro compound (IIIa) to form an amino compound (IVa) 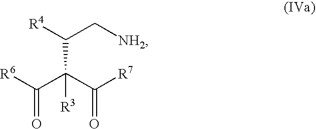
followed by an intramolecular cyclization reaction to form a compound (Va)

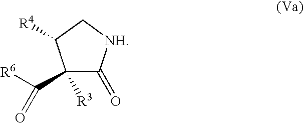

17. The method of claim 16 wherein compound (IIIa) structure 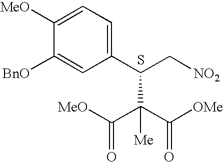
wherein Me is methyl and Bn is benzyl.

18. The method of claim 16 wherein compound (IIIa) has a structure 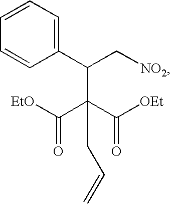
wherein Et is ethyl.

19. The method of claim 16 wherein compound (Va) has a structure 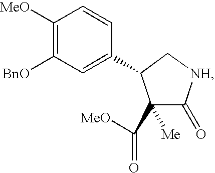
wherein Me is methyl and Bn is benzyl.

20. (canceled)

21. A compound having a structural formula (III) 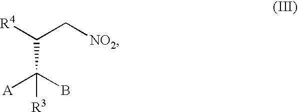
wherein A is selected from the group consisting of C(═O)OR¹, C(═O)N(R⁵)₂, C(═O)SR⁵, CN, NO₂, and SO₂R⁵; B is selected from the group consisting of C(═O)OR², C(═O)N(R⁵)₂, C(═O)SR⁵, and CN; R¹ is selected from the group consisting of C₁₋₄alkyl, hydro, and M; R² is selected from the group consisting of hydro, M, alkoxyalkyl, alkyl, cycloalkyl, aryl, C₁₋₃alkylenearyl, heteroaryl, and C₁₋₃alkyleneheteroaryl; R³ is selected from the group consisting of C₁₋₄alkyl, alkoxy, acylamino, halo, alkylthio, allyl, C₁₋₃alkylenearyl, and cyanoC₁₋₃alkyl; R₄ is selected from the group consisting of unsubstituted or substituted aryl and heteroaryl; R⁵, independently, is selected from the group consisting of hydro, C₁₋₄alkyl, cycloalkyl, aryl, C₁₋₃alkylenearyl, heteroaryl, and C₁₋₃alkyleneheteroaryl; and M is an alkali metal cation or an alkaline earth metal cation;
said compound (III) prepared by a method comprising reacting a compound having a structural formula (I) 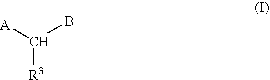
with a nitroolefin of structural formula (II), 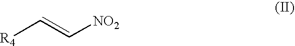
said reaction performed in the presence of a base and a catalyst complex comprising a ligand and a metal complex.

22. A compound having a structural formula (V) 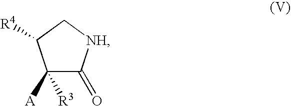
wherein A is selected from the group consisting of C(═O)OR¹, C(═O)N(R⁵)₂, C(═O)SR⁵, CN, NO₂, and SO₂R⁵; R¹ is selected from the group consisting of C₁₋₄alkyl, hydro, and M; R³ is selected from the group consisting of C₁₋₄alkyl, alkoxy, acylamino, halo, alkylthio, allyl, C₁₋₃alkylenearyl, and cyanoC₁₋₃alkyl; R₄ is selected from the group consisting of unsubstituted or substituted aryl and heteroaryl; R⁵, independently, is selected from the group consisting of hydro, C₁₋₄alkyl, cycloalkyl, aryl, C₁₋₃alkylenearyl, heteroaryl, and C₁₋₃alkyleneheteroaryl; and M is an alkali metal cation or an alkaline earth metal cation;
said compound (V) prepared by a method comprising the steps of:
(a) reacting a compound of structural formula (I) 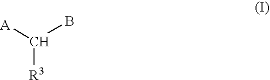
wherein B is selected from the group consisting of C(═O)OR², C(═O)N(R⁵)₂, C(═O)SR⁵, CN, and NO₂; and R² is selected from the group consisting of hydro, M, alkoxyalkyl, alkyl, cycloalkyl, aryl, C₁₋₃alkylenearyl, heteroaryl, and C₁₋₃alkyleneheteroaryl;
with a nitroolefin of structural formula (II) 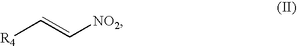
said reaction performed in the presence of a base and a catalyst complex comprising a ligand and a metal complex to form a compound having a structural formula (III) 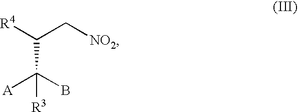
(b) converting the nitro group of compound (III) to form an amino compound (IV) 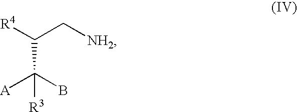
followed by (c) an intramolecular cyclization reaction to form the compound (V).

23. A compound having a structural formula (IIIa) 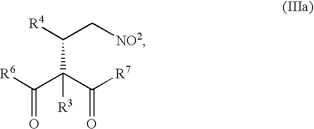
wherein R⁶ is alkoxy, amino, or thio; R⁷ is selected from the group consisting of alkoxy, alkoxyalkyl, alkyl, cycloalkyl, aryl, C₁₋₃alkylenearyl, heteroaryl, and C₁₋₃alkyleneheteroaryl; R³ is selected from the group consisting of C₁₋₄alkyl, alkoxy, acylamino, halo, alkylthio, allyl, C₁₋₃alkylenearyl, and cyanoC₁₋₃alkyl; and R⁴ is selected from the group consisting of unsubstituted or substituted aryl and heteroaryl;
said compound (IIIa) prepared by a method comprising the step of reacting an α-substituted β-dicarbonyl compound of structural formula (Ia) 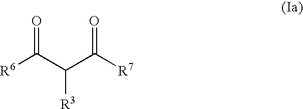
with a nitroolefin of structural formula (II), 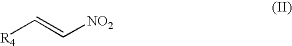
said reaction performed in the presence of a base and a catalyst complex comprising a ligand and a metal complex.

24. A compound having a structural formula (Va) 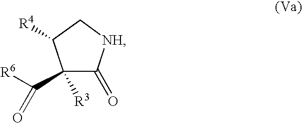
wherein R⁶ is alkoxy, amino, or thio; R³ is selected from the group consisting of C₁₋₄alkyl, alkoxy, acylamino, halo, alkylthio, allyl, C₁₋₃alkylenearyl, and cyanoC₁₋₃alkyl; and R⁴ is selected from the group consisting of unsubstituted or substituted aryl and heteroaryl;
said compound (Va) prepared by a method comprising the steps of:
(a) reacting an α-substituted β-dicarbonyl compound of structural formula (Ia) 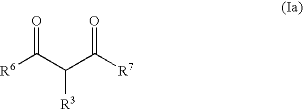
wherein R⁷ is selected from the group consisting of alkoxy, alkoxyalkyl, alkyl, cycloalkyl, aryl, C₁₋₃alkylenearyl, heteroaryl, and C₁₋₃alkyleneheteroaryl;
with a nitroolefin of structural formula (II) 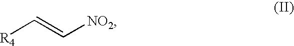
said reaction performed in the presence of a base and a catalyst complex comprising a ligand and a metal complex to form a compound having a structural formula (IIIa) 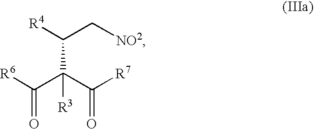
(b) converting the nitro group of compound (IIIa) to form an amino compound (IVa) 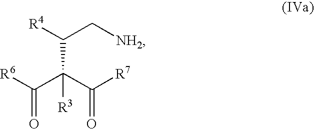
followed by (c) an intramolecular cyclization reaction to form the compound (Va).
